(19) United States
(12) Patent Application Publication
(10) Pub. No.: US 2008/0103207 A1
(43) Pub. Date: May 1, 2008

(54) TOPICAL CERAMIDE COMPOSITIONS AND METHODS OF USE

(75) Inventor: Nava Dayan, Fair Lawn, NJ (US)

Correspondence Address: DARBY & DARBY P.C., P.O. BOX 770, Church Street Station, New York, NY, 10008-0770, US

(73) Assignees: Takasago International Corp. (USA), Rockleigh, NJ (US); Lipo Chemicals Inc., Paterson, NJ (US)

(21) Appl. No.: 11/835,322

(22) Filed: Aug. 7, 2007

Related U.S. Application Data
(60) Provisional application No. 60/821,890, filed on Aug. 9, 2006.

(51) Int. Cl. A61K 31/16

(52) U.S. Cl. 514/613

(57) ABSTRACT

Ceramide NS and butylene glycol can form a topical composition in which the ceramide NS is lamellar. The topical compositions can be used in a formulation such as a lotion, gel, or cream and can be used to treat an aging-related skin condition, dry skin, skin irritation, skin wrinkles, or other conditions in which the skin barrier is compromised, damaged, or disordered. These compositions can manufactured by dispersing ceramide NS in butylene glycol while heating and stirring.

### 1. CROSS REFERENCE TO RELATED APPLICATIONS

Priority is claimed under 35 U.S.C. 119(e) to copending U.S. provisional patent application Ser. No. 60/821,890 filed on Aug. 9, 2006. The contents of this prior application are hereby incorporated by reference and in their entirety.

### 2. FIELD OF THE INVENTION

The present invention provides topical compositions comprising lamellar ceramide NS and butylene glycol. Topical compositions of the invention can be used in a formulation such as a gel or cream and can be used to treat an aging-related skin condition, dry skin, skin irritation, skin wrinkles, or other conditions in which the skin barrier is compromised, damaged, or disordered. The compositions of the invention can manufactured by dispersing ceramide NS in butylene glycol while heating and stirring.

### 3. BACKGROUND OF THE INVENTION

Although the stratum corneum is less than 10% of the skin, more than 80% of the skin's permeability barrier function is derived from this layer of the epidermis. The stratum coreum is composed of comeocytes surrounded by a matrix of lipid bilayers which is composed of mainly fatty acids, cholesterol, and ceramides. The matrix is mostly in a solid, non-permeable gel state with some domains existing in a permeable, liquid crystalline state.

Ceramides are simple sphingolipids and are composed of sphingosine (an 18-carbon chain with hydroxyl and amine groups) with an amide linked fatty acid. Ceramides are also one of the most hydrophobic molecules in nature making the permeability of ceramides applied to the skin low. The nine classes of ceramides (ceramide 1 to ceramide 9) are essential for multiple biological processes including apoptosis, signal transduction, and mitosis and play a key role in the barrier function of the stratum corneum including trans epidermal water loss (TEWL). Ceramide 2 (or ceramide NS) is derived from epidermal sphingomyelin SM-1 and has been shown to inhibit cell proliferation and induce apoptosis. U.S. Pat. No. 6,355,232 discloses a method to synthesize 99% optically pure ceramide NS (2S, 3R) which is the isomer found in the body, its solubility profile in selected solvents, and its ability to act as a water barrier.

As the skin ages, the total lipid content in the skin decreases by 30%. The proportion of skin lipids also changes. A decrease in the total ceramide content during aging results in skin disorders caused by the diminished barrier function of the skin. Formulations containing ceramides, including ceramide 2, can improve these skin conditions.

Ceramide 2 is found in cosmetic formulations in a variety of combinations that allows its incorporation into creams and lotions. For example, ceramide 2 can be incorporated into a composition with C12-15 alkyl benzoate, tribehenin, PEG-10 rapeseed sterol and palmitoyl oligopeptide (the composition is also known as Dermaxyl®). Ceramide 2 can also be incorporated into a composition with water, alcohol, cholesterol, hydrogenated lecithin, ceramide 3, palmitic acid and oleic acid (the composition is also known as cerasome). Ceramide 2 can also be incorporated into a composition with PEG-60 hydrogenated castor oil and PEG-8 (the composition is also known as ceramide 2 sol 2%). However, none of these compositions contain butylene glycol and none have been tested for their bioavailability. Furthermore, these compositions contain ceramide 2 that is a mixture of different isomers.

Thus, there continues to be a need in the art to identify compositions containing a high percentage of the natural isomer of ceramide NS (ceramide NS (2S,3R)) in its lamellar, bioavailable form. These compositions can be applied topically such that the lamellar ceramide NS is incorporated into the stratum corneum to increase the barrier function of the skin.

### 4. SUMMARY OF THE INVENTION

The present invention includes a composition comprising ceramide NS and butylene glycol. The composition can include ceramide NS that is lamellar. The ceramide NS of the composition can be in the form of the isomer, ceramide NS (2S, 3R). The composition can be anhydrous and include ceramide in an amount up to about 5% (wt ceramide/vol butylene glycol) of the composition. The composition can be combined with a pharmaceutically acceptable excipient to form a topical formulation such as a cream, lotion, or gel.

Methods of making the composition are also included in the present invention. These methods include dispersing the ceramide NS powder in butylene glycol and heating the dispersion with constant stirring. Methods of treating an aging-related skin condition, dry skin, skin irritation, skin wrinkles, a condition in which the skin barrier is compromised, damaged, or disordered, or combination thereof in a subject comprising administering a pharmaceutically acceptable amount of the composition are also included in the present invention. Further methods of strengthening skin, firming skin, rejuvenating skin, or restoring the condition of the skin in a subject comprising administering a pharmaceutically acceptable amount of the composition are also included. For example, the present invention includes a method of treating skin irritation after insult in menopausal women comprising administering a pharmaceutically acceptable amount of the composition.

The above features and many other attendant advantages of the invention will become better understood by reference to the following detailed description when taken in conjunction with the accompanying drawings.

### 5. BRIEF DESCRIPTION OF THE DRAWINGS

FIG. 1 is a chiral HPLC chromatograph of ceramide NS (2S, 3R). The chromatograph was obtained by adding 20 mL of THF to 50 mg of ceramide NS (2S, 3R) and heating the mixture to dissolve. After cooling, THF was added to the solution to make a total of 50 mL. Two mL of the resulting solution were combined with a mixture of n-hexane and ethanol (95:5 (vol:vol)) to obtain a total of 10 mL of test solution. Ten microliters of test solution were analyzed by HPLC using the following conditions:
Detector: UV absorption spectrophotometer (210 nm)
Column: stainless tube (inside diameter 4 mm×about 25 cm) SUMICHIRAK® OA-4600 (4.6 mm×25 cm, Sumika Chemical Analysis Service, Ltd., Osaka, Japan)
Column 5 micron aminopropyl silica gel which is covalently bonded to N-Stationary [(S)-1-(alpha-naphthyl)ethylaminocarbonyl]-L-tert-leucine Phases:
Temperature of 25 degrees C. Column:
Mobile Phase: mixture of n-hexane and ethanol (95:5 (vol:vol))
Flow Rate: adjusted such that the retention time of ceramide NS (2S, 3R) is about 14 minutes
Optical Purity (%)=(area for peak at about 14 minutes/total area for all peaks between 10 and 20 minutes)×100

FIG. 2 is transmission electron micrographs of (A) 0.05% (wt/vol) of 97 wt % ceramide NS (2S,3R) dispersed in butylene glycol and (B) 0.05% (wt/vol) of 97 wt % ceramide NS (2S,3R) dispersed in pentylene glycol.

### 6. DETAILED DESCRIPTION OF THE INVENTION

The present invention is based on the surprising discovery that ceramide NS (2S,3R) can be solubilized in butylene glycol, but can not be solubilized in other solvents, such as pentylene glycol. In this system, ceramide NS exists in a non-crystalline, lamellar state.

Ceramide NS in a lamellar state is highly desirable for use in topical formulations. Unlike crystalline ceramide NS, lamellar ceramide NS is bioavailable and, thus, interacts effectively with the intracellular lipids of the stratum corneum to increase the barrier function of the skin. Ceramide NS is also the only ceramide known to be converted into other ceramides. Lamellar, isometrically pure ceramide NS (2S, 3R) topically administered to the skin can serve as an efficiently recognized enzymatic substrate for conversion to other ceramides such as ceramides 8, 5, and 7 (also known as ceramide NH, AS, AH, respectively). Thus, without being bound by any particular theory, compositions containing lamellar ceramide NS as opposed to other ceramides are highly desirable because the creation of a variety of ceramides may lead to a faster skin barrier repair, inhibition of cell proliferation, induction of apoptosis, and, consequently, promotion of the health of the skin.

The terms “about” or “approximately” mean within an acceptable range for the particular parameter specified as determined by one of ordinary skill in the art, which will depend in part on how the value is measured or determined, e.g., the limitations of the measurement system. For example, “about” can mean a range of up to 20% of a given value. Alternatively, particularly with respect to biological systems or processes, the term can mean within an order of magnitude, preferably within 5-fold, and more preferably within 2-fold, of a value.

6.1. Compositions Comprising Lamellar Ceramide NS and Butylene Glycol

Compositions of the present invention contain ceramide NS (also known as ceramide 2) in lamellar form and butylene glycol.

The ceramide NS used in the present invention is present in an amount to be lamellar in a composition containing butylene glycol and is preferably ceramide NS (2S, 3R) which is the natural isomer of ceramide NS (also referred to herein as “nature identical ceramide NS”). Ninety-seven % pure ceramide NS (2S, 3R) has a chiral HPLC chromatograph provided in FIG. 1 and is available from Takasago International Corporation (Rockleigh, N.J.). See also Dayan and Wertz (Intl. Symp. Cont. Rel. Austria, Proceeding #980, 2006) discussing the isomerical purity of two synthetic ceramide NS preparations.

In butylene glycol, the lamellar ceramide NS can be present in an amount up to and including about 5% (wt/vol), preferably from up to and including about 2% (wt/vol), and more preferably from up to and including about 1.5% (wt/vol). In butylene glycol, the lamellar ceramide NS can also be present in an amount from about 0.001% (wt/vol) to about 5% (wt/vol), from about 0.001% (wt/vol) to about 2% (wt/vol), from about 0.5% (wt/vol) to about 5% (wt/vol), and from about 0.5% (wt/vol) to about 2% (wt/vol).

The lamellarity of ceramide NS can be observed using transmission electron microscopy (TEM). For example, preparations of ceramide NS in butylene glycol can be observed using TEM after negative staining by mounting a drop of diluted preparation onto a copper grid, applying phosphotungstic acid (PTA) or uranyl acetate (UA) negative stain, and drying the grid preparation. The prepared specimen can be observed under a TEM at 100 kv-accelerated voltage.

Butylene glycol, preferably 1,3 butylene glycol, is used in the composition with ceramide NS. 1,3-Butylene glycol is available from Ruger Chemicals (Linden N.J.). 1,2-Butylene glycol, 1,4 butylene glycol, and 2,3 butylene glycol are available from Sigma-Aldrich (St. Louis, Mo.). Butylene glycol can be present in a composition of the invention in an amount sufficient such that the ceramide NS of the composition is lamellar. In a solution containing ceramide NS, butylene glycol can be present in an amount greater than or equal to about 95% (wt/vol), preferably greater than or equal to about 98% (wt/vol), and more preferably greater than or equal to about 98.5% (wt/vol). In a solution containing ceramide NS, butylene glycol can also be present in an amount from about 95% (wt/vol) to about 99.999% (wt/vol), from about 98% (wt/vol) to about 99.999% (wt/vol), from about 95% (wt/vol) to about 99.5% (wt/vol), and from about 98% (wt/vol) to about 99.5% (wt/vol).

A composition comprising lamellar ceramide NS and butylene glycol of the present invention is preferably anhydrous. Anhydrous compositions containing ceramide NS are desirable since they do not require preservation.

6.2. Methods of Making Compositions Comprising Lamellar Ceramide NS and Butylene Glycol

The composition containing lamellar ceramide NS and butylene glycol can be made by dispersing ceramide NS powder in butylene glycol and heating the dispersion to a range of temperatures from about 50 to about 85 degrees Celsius, and preferably from about 70 to about 80 degrees Celsius, with constant stirring.

6.3. Topical Formulations Containing a Composition Comprising Lamellar Ceramide NS and Butylene Glycol and a Pharmaceutically Acceptable Excipient

A therapeutically effective amount of the composition comprising lamellar ceramide NS and butylene glycol can be added to the oil phase of a topical formulation. For example, when present in a therapeutic or pharmaceutical formulation, the amount of lamellar NS can be in the range of about 0.001% (wt/vol) to about 2% (wt/vol) of the formulation and preferably in the range of about 0.01% (wt/vol) to about 0.50% (wt/vol) of the formulation. Lamellar ceramide NS in butylene glycol can also be added to the water phase of a formulation or can be added at about 40 degrees to about 45 degrees Celsius during the cool-down procedure of a formulation.

Topical formulations can be in, for example, the form of a solution, suspension, gel, paste, balm, cream, lotion, leave-on exfoliating product, eye treatment, scalp treatment, daily wear moisturizer, sunscreen, after-sun product, and hair product, with or without an additional active agent. Topical formulations can be manufactured using techniques well know to one of ordinary skill in the art.

Pharmaceutically acceptable diluents, auxiliary agents, and excipients for topical use are well known in the pharmaceutical and cosmetic field, and are described, for example, in Remington's Pharmaceutical Sciences, Mack Publishing Co., (A. R. Gennaro edit. 1995). The materials are nontoxic to a recipient at the dosages and concentrations employed, and include buffers such as phosphate, citrate, acetate or other organic acid salts; antioxidants such as ascorbic acid; low molecular weight (less than about ten residues) peptides such as polyarginine; proteins such as serum albumin, gelatin, or immunoglobulins; hydrophilic polymers such as polyvinylpyrrolidinone; natural or synthetic oils, including vegetable oil; wax; glycerine; amino acids such as glycine, glutamic acid, aspartic acid, or arginine; monosaccharides, disaccharides, and other carbohydrates including cellulose or its derivatives, glucose, lactose, mannose or dextrins; chelating agents such as EDTA; sugar alcohols such as mannitol or sorbitol; salts such as sodium chloride; nonionic surfactants such as Tween®, Pluronics® or polyethyleneglycol; and other detergents. Other suitable excipients or carriers are described throughout the present disclosure including the examples.

Additional active agents that can be added to the formulation include alpha hydroxyl acids (AHAs), lactic acid, beta hydroxyl acids (BHAs) such as salicylic acid, skin brightening agents such as kojic acid, hydroxyl cinnannic acid, licorice extract, other plant extracts, retinyl palmitate, Coenzyme Q10, vitamin A, vitamin E, avobenzone, octinoxate, octisalate, oxybenzone, or other agents that treat aging.

A topical formulation of the present invention can also include ascorbic acid and its derivatives, ursolic acid, nicotinamide, other compounds that induce the production of ceramides, or combinations thereof. Additional glycols can also be included in a formulation of the present invention.

The topical formulation can also be a skin covering or dressing containing a therapeutically effective amount of lamellar ceramide NS impregnated into, covalently attached, or otherwise associated with a covering or dressing material. The skin covering or dressing can permit release of the composition. Release of the composition can be in an uncontrolled or a controlled manner. Hence, the skin coverings or wound dressings of the invention can provide slow or timed release of the composition onto the skin. Skin coverings and dressing materials can be any material used in the art, for example, bandage, gauze, sterile wrapping, hydrogel, hydrocolloid and similar materials.

6.4. Treatment Methods

A therapeutically effective amount of a composition containing lamellar ceramide NS and butylene glycol can be administered to a subject in need thereof to treat:

(a) an aging-related skin condition,

(b) dry skin,

(c) skin irritation,

(d) skin wrinkles,

(e) other conditions in which the skin barrier is compromised, damaged, or disordered, or

(f) combination thereof.

As used herein, the terms “treat,” “treating,” or “treatment” mean the prevention, reduction, amelioration, partial or complete alleviation, or cure.

An aging-related skin condition is any skin condition or disorder associated with, caused by, or affected by, intrinsic aging and/or extrinsic aging. Aging-related skin conditions that may be treated using the present methods and compositions include, but are not limited to, wrinkles, age spots, sun damage (such as UV radiation-induced oxidative stress), blemishes, hyperpigmented skin, age spots, increased skin thickness, loss of skin elasticity and collagen content, dry skin, xerosis, lentigines, and melasmas.

The present invention also provides compositions containing lamellar ceramide NS and butylene glycol that are useful as agents to treat skin-related conditions, such as dry skin, skin irritation, wrinkles, burns, atopic dermatitis, psoriasis, wounded skin, or other conditions in which the skin barrier is compromised, damaged, or disordered. These compositions are useful in strengthening skin, firming skin, rejuvenating skin, and restoring the condition of the skin. These compositions are also useful as agents to treat photodamaged skin.

Examples of subjects with conditions that can be treated using a composition containing lamellar ceramide NS and butylene glycol include the elderly, menopausal women, and burn victims.

One particular method of the invention is a method of treating skin irritation after insult, such as after shaving or skin wounding, in menopausal women comprising administering a pharmaceutically effective amount of a composition containing lamellar ceramide NS and butylene glycol.

6.4.1. Method of Administration

A therapeutically effective amount of the composition is an amount of the composition that increases ceramide NS in the skin to a degree needed to maintain healthy skin, to promote the condition of the skin, or both. A therapeutically effective amount of the composition can also be an amount of the composition that increases ceramide NS in the skin to a degree needed to treat skin-related conditions, such as wrinkles, dry skin, and skin irritation. Further, a therapeutically effective amount of the composition can be an amount of the composition that increases the amount of ceramide NS in the skin to a degree needed to strengthen or firm skin, rejuvenate and/or restore the condition of the skin, for example, to restore the condition of photodamaged skin.

The therapeutically effective amount of the composition can vary with the type of topical administration. However, the amount of the composition required for healthy skin development may vary not only with the route of administration, but also the nature of the condition being treated, and the age and condition of the patient, and will be ultimately at the discretion of the attendant physician or clinician.

The dose and method of administration can vary depending upon the location of the skin to be treated. The composition can be topically administered and can contain from about 0.001% (wt/vol) to about 2% (wt/vol) and preferably from about 0.01% (wt/vol) to about 0.50% (wt/vol) of lamellar ceramide NS. The desired dose may be presented in a single dose, as divided doses, or as a continuous infusion into the skin. The desired dose can also be administered at appropriate intervals, for example, as two, three, four or more sub-doses per day for at least 6 weeks, preferably at least 12 weeks, and most preferably at least 8 weeks. The formulation can also be administered indefinitely. One of skill in the art can readily prepare and administer an effective formulation from available information using the teachings provided herein.

6.4.2. Assays to Determine Effectiveness of a Method of Treatment

Examples of assays to measure the effectiveness of a method of treatment of the invention include measurement of trans epidermal water loss (TEWL), skin conductance, and erythema.

TEWL (i.e., the amount of water vapor lost across the stratum coreum) can be measured using an evaporimeter, e.g, Evaporimeter EP-2™ (available from ServoMed, Sweden) or a research grade evaporimeter (available from cyberDERM in Broomall, Pa.). Normal TEWL values are between about 2 and about 5 g/m² per hour. TEWL values can reach values as high as about 90 to about 100 g/m² per hour after skin stripping or in the case of an aging-related skin condition.

As shown by Obata and Tagami (J. Soc. Cosmet. Chem. 1990, 41:235-241), the ability of an alternating current to flow through the stratum corneum is an indirect measure of its water content. Skin conductance can be measured using a skin surface hygrometer (e.g., the SKICON-200EX available from I.B.S Co., Ltd., Japan) that uses a high frequency current to measure skin surface moisture. After skin stripping or in the case of an aging-related skin condition, skin conductance can be reduced compared to normal skin.

Skin erythema can be measured by ranking the erythema on a scale from 0 (none) to 8 (marked erythema, edema, possible erosion). Skin erythema measurements can also be quantified by measuring the changes in skin redness based on the amount of light reflected (measured in luxes) from the skin surface from a known amount of illuminated light delivered to the skin surface. A reduction in the luminous flux from the skin surface is used as an indicator of a darker skin surface as a result of an increased skin blood flow within the measurement region.

### 7. EXAMPLE 1

### Making a Composition Containing Lamellar Ceramide NS and Butylene Glycol

0.05% (wt/vol) of 97% Ceramide NS (2S, 3R) powder (available from Takasago International Corporation, Rockleigh, N.J.) was dispersed in butylene glycol and heated to 70 degree Celsius with constant stirring.

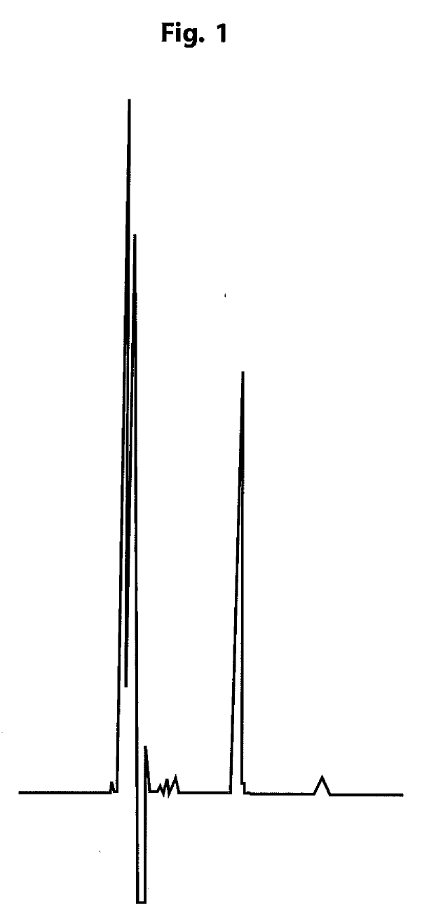

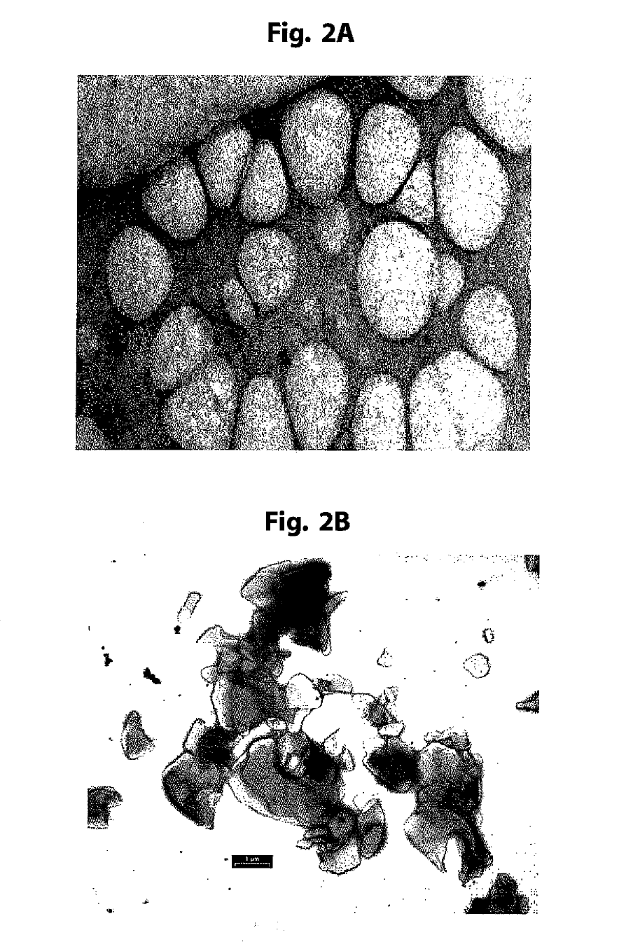

As observed using negative staining TEM, the ceramide NS in the resulting mixture had a round lamellar structure as shown in FIG. 2A. The structure of round lamellar ceramide NS can be contrasted with crystalline ceramide NS in pentylene glycol at the same concentration made by the same method as shown in FIG. 2B.

### 8. EXAMPLE 2

### Composition containing Lamellar Ceramide NS and Butylene Glycol

A rich moisturizing eye cream that hydrates the delicate skin around the eyes can be made using lamellar ceramide NS and butylene glycol as described below. Daily application of the cream aids in reducing the appearance of fine lines to produce plump, healthy, and hydrate skin.

The formulation:

| | | | International |
|---|---|---|---|
| | Percent | Ingredient | Name of Cosmetic |
| Se- | (as wt | (supplier name | Ingredients |
| quence | or vol) | and location) | (INCI) Name |
| 1 | 50.55 | deionized water | water |
| 1 | 15.00 | Keltrol ® (1%) | xanthan gum |
| | | (Kelco ®, | |
| | | Minneapolis MN) | |
| 1 | 1.00 | Liposerve ™ PP | phenoxyethanol, |
| | | (Lipo Chemicals | methylparaben, |
| | | Inc., Paterson, NJ) | ethylparaben, |
| | | | butylparaben, |
| | | | propylparaben, and |
| | | | isobutylparaben |
| 1 | 10.00 | Veegum ® HV (4%) | magnesium |
| | | (RT Vanderbuilt, | aluminum silicate |
| | | Norwalk, CT) | |
| 2 | 5.00 | Dow Corning ® 9040 | cyclopentasiloxane |
| | | (Dow Corning ®, | and dimethicone |
| | | Midland, MI) | crosspolymer |
| 2 | 4.00 | Panalane ® L-14E | hydrogenated |
| | | (Lipo (Ineos), | polyisobutene |
| | | League City, TX) | |
| 2 | 2.00 | Lipocol ® SC (Lipo | cetearyl alcohol |
| | | Chemicals Inc., | |
| | | Paterson, NJ) | |
| 2 | 1.00 | Dow Corning 345 | cyclopentasiloxane |
| | | Fluid (Dow | |
| | | Corning ®, Midland, MI) | |
| 2 | 1.00 | Lipocol ® S-2 (Lipo | steareth-2 |
| | | Chemicals Inc., | |
| | | Paterson, NJ) | |
| 2 | 0.75 | Liposorb ® L-20 | polysorbate 20 |
| | | (Lipo Chemicals | |
| | | Inc., Paterson, NJ) | |
| 2 | 1.20 | Lipovol ® GTB | tribehenin |
| | | (Lipo Chemicals | |
| | | Inc., Paterson, NJ) | |
| 3 | 2.50 | Simulgel ® NS | hydroxyethyl |
| | | (Fairfield, NJ) | acrylate/sodium |
| | | | acryloyldimethyl |
| | | | taurate copolymer, |
| | | | squalane, |
| | | | polysorbate 60 |
| 4 | 5.00 | 2% (wt/vol) | butylene glycol and |
| | | ceramide NS in | ceramide 2 |
| | | butylene glycol | |

The eye cream was made by

(1) combining Sequence #1 ingredients and heating to 78-80 degrees Celsius with propeller mixing,

(2) heating Sequence #2 to 80 degrees Celsius and mixing until uniform,

(3) adding Sequence #2 to Sequence #1 with medium speed propeller mixing,

(4) cooling the batch to 50 degrees Celsius,

(5) adding sequence #3 to the batch and increasing the mixing speed as the batch begins to thicken,

(6) adding sequence #4 to the batch and mixing well, and

(7) cooling to 25 degrees Celsius.

The specifications for the eye cream are:
pH: 7.2-7.6
viscosity: LVT #4 (6 rpm 71,000±10%
stability: 30 days at 50 degrees Celsius

### 9. EXAMPLE 3

### The Treatment with Lamellar Ceramide NS Showed Potential Improvement in Skin Irritation after Insult in Menopausal Women

9.1. Materials and Methods

0.5 wt % of 97% nature identical ceramide NS was dispersed in butylene glycol and heated to 70 degrees Celsius with constant stirring to produce the test product.

A randomized, double blind, vehicle controlled study was performed using this composition as follows. Fourteen women between the ages of 47-69 and at least 1 year post-menopausal were divided into two groups (vehicle and test product). The women were subjected to tape stripping, a form of skin barrier insult, in which a total of 16 tape strips were applied and removed repeatedly from each volar forearm using Leukoflex® tape (available from Smith & Nephew Pty Limited, Mount Waverly, Victoria, Australia). Following tape stripping, the women applied either the vehicle or test product topically twice daily for eight weeks to a portion of both their intact and insulted skin. Butylene glycol was used as the vehicle.

Measurements of TEWL, erythema, and skin conductance were taken on the intact and the insulted skin of the women at 2 week intervals during the course of the 8 week treatment period.

TWEL was measured using a research grade evaporimeter (available from cyberDERM in Broomall, Pa.) equipped with TEWL probes that were manufactured by Cortex Technology (Hadsund, Denmark). This instrument is based on the vapor pressure gradient estimation method (Grove et al., Skin Res. And Tech. 1999, 1-8). TWEL measurements that were taken post stripping, but prior to test application, were performed approximately 30 minutes after tape stripping.

Erythema was measured by grading erythema on a scale from 0 (none) to 8 (marked erythema, edema, possible erosion).

Skin conductance was measured using a skin surface hygrometer (SKICON-200EX available from I.B.S Co., Ltd., Japan) equipped with a Measurements Technologies probe (Dayton, Ohio) to enhance further its ability to measure changes in skin surface hydration.

9.2. Results

TEWL was measured by mean water loss (g/m² per hour) at baseline (i.e., before application of a composition) and at 2, 4, 6 and 8 weeks after application of either the 0.5% (wt/vol) ceramide NS composition in the vehicle or the vehicle to the skin. The TEWL of untreated skin was also measured. As shown in Table 1, the application of the lamellar ceramide NS composition reduced TEWL compared to application of only the vehicle which is a skin penetration enhancer. The increase in TEWL of ceramide NS-treated skin above the TEWL of vehicle-treated skin at week 8 may reflect a reduced patient compliance between weeks 6 and 8.

TABLE 1
TWEL (mean water loss in g/m² per hour) following
the Application of Ceramide NS in the Vehicle or
the Vehicle to Skin compared to Untreated Skin

| | Ceramide | | |
|---|---|---|---|
| | NS | Vehicle | Untreated |
| Baseline | 4.37 | 4.45 | 4.37 |
| Week 2 | 5.03 | 5.72 | 4.71 |
| Week 4 | 5.8 | 6.12 | 4.97 |
| Week 6 | 5.52 | 5.91 | 4.91 |
| Week 8 | 6.07 | 6.01 | 5.3 |

Additionally, skin erythema scores were taken at baseline (i.e., before application of a composition) and at 2, 4, 6 and 8 weeks after application of either the 0.5% (wt/vol) ceramide NS composition in the vehicle or the vehicle to the skin. The skin erythema scores of untreated skin were also measured. As shown in Table 2, the application of the lamellar ceramide NS composition reduced skin erythema compared to skin with only the vehicle applied or untreated skin.

TABLE 2
Skin Erythema Scores following the Application of Ceramide NS in
the Vehicle or the Vehicle to Skin compared to Untreated Skin

| | Ceramide | | |
|---|---|---|---|
| | NS | Vehicle | Untreated |
| Baseline | 0 | 0 | 0 |
| Week 2 | 0.67 | 1.2 | 0.82 |
| Week 4 | 0.19 | 0.33 | 0.39 |
| Week 6 | 0.31 | 0.76 | 0.54 |
| Week 8 | 0.33 | 1.04 | 0.74 |

Furthermore, the skin conductivity (in microohms) was measured at baseline (i.e., before application of a composition) and at 2, 4, 6 and 8 weeks after application of either the 0.5% (wt/vol) ceramide NS composition in the vehicle or the vehicle to the skin. The skin conductivities of untreated skin were also measured. As shown in Table 3, skin conductance compared to baseline was consistently higher in skin treated with the lamellar ceramide NS composition compared to untreated skin and skin treated with the vehicle.

TABLE 3
Skin Conductivity (in microohms) relative to baseline
following the Application of Ceramide NS in the Vehicle
or the Vehicle to Skin compared to Untreated Skin

| | Ceramide | | |
|---|---|---|---|
| | NS | Vehicle | Untreated |
| Week 2 | 316.86 | 291.83 | 282.63 |
| Week 4 | 360.37 | 314.09 | 321.7 |
| Week 6 | 297.49 | 248.54 | 248.57 |
| Week 8 | 243.71 | 214.66 | 210.36 |

Thus, the lamellar ceramide NS composition was shown to reduce TEWL and skin erythema levels while elevating skin conductivity.

TEWL as measured by mean water loss (g/m² per hour) was measured for skin without insult (i.e., baseline), for skin after tape stripping but before application of the lamellar ceramide NS composition, and for skin after tape stripping and 2 weeks after twice daily application of the lamellar ceramide NS composition. Table 4 demonstrates that, although the TEWL reading following tape stripping prior application of the lamellar ceramide NS composition was elevated compared to both untreated skin and skin treated with only vehicle, after two weeks of twice daily application of the lamellar ceramide NS composition, TEWL was lowest in skin treated with the lamellar ceramide NS composition when compared to untreated, tape stripped skin and tape stripped skin treated with only vehicle. Similar results were demonstrated when measuring skin conductivity.

TABLE 4
TEWL (mean water loss in g/m² per hour) for skin without insult (i.e.,
baseline), for skin after tape stripping but before application of
the lamellar ceramide NS composition (i.e., Tape Stripped), and for
skin after tape stripping and 2 weeks after twice daily application
of the lamellar ceramide NS composition (i.e., Week 2)

| | Ceramide | | |
|---|---|---|---|
| | NS | Vehicle | Untreated |
| Baseline | 4.37 | 4.45 | 4.37 |
| Tape | 13.17 | 11.96 | 9.68 |
| Stripped | | | |
| Week 2 | 5.45 | 6.62 | 9.88 |

Skin erythema of skin after tape stripping followed by two weeks of twice daily application of the lamellar ceramide NS composition, in skin after tape stripping followed by two weeks of twice daily application of the vehicle, and in skin two weeks after tape stripping was measured. The application of the lamellar ceramide NS composition to skin twice daily for two weeks after tape stripping reduced skin erythema by 73% compared to a 69% reduction in skin treated with vehicle twice daily for two weeks after tape stripping and a 69% reduction in untreated skin two weeks after tape stripping.

In sum, the lamellar ceramide NS composition was shown to improve the skin barrier repair rate after insult as reflected in the low TEWL and skin erythema values of skin treated with the lamellar ceramide NS composition compared to vehicle-treated or untreated skin.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein, including all patents, published patent applications, and published scientific articles, are incorporated by reference in their entireties for all purposes.

What is claimed is:

1. A composition comprising ceramide NS and butylene glycol.

2. The composition of claim 1, wherein the ceramide NS is lamellar.

3. The composition of claim 1, wherein the composition is anhydrous.

4. The composition of claim 1, wherein the ceramide NS is ceramide NS (2S,3R).

5. The composition of claim 1, wherein the ceramide NS is present in an amount up to about 5% (wt ceramide/vol butylene glycol) of the composition.

6. A topical formulation comprising the composition of claim 1 and a pharmaceutically acceptable excipient.

7. The topical formulation of claim 6, wherein the formulation is a cream, lotion, or gel.

8. A method of making the composition of claim 1 comprising dispersing ceramide NS powder in the butylene glycol and heating the dispersion to from about 70 to about 80 degrees Celsius with constant stirring.

9. A method of treating an aging-related skin condition, dry skin, skin irritation, skin wrinkles, a condition in which the skin barrier is compromised, damaged, or disordered, or combination thereof in a subject comprising administering a pharmaceutically acceptable amount of the composition of claim 1 to the subject.

10. A method of treating skin irritation after insult in menopausal women comprising administering a pharmaceutically acceptable amount of the composition to claim 1.

11. A method of strengthening skin, firming skin, rejuvenating skin, or restoring the condition of the skin in a subject comprising administering a pharmaceutically acceptable amount of the composition to claim 1 to the subject.
